(19) United States
(12) Patent Application Publication
(10) Pub. No.: US 2007/0003612 A1
(43) Pub. Date: Jan. 4, 2007

(54) Capsule

(75) Inventor: Lyndsay Williams, Cambridge (GB)

Correspondence Address: MICROSOFT CORPORATION;ATTN: PATENT GROUP DOCKETING DEPARTMENT, ONE MICROSOFT WAY, REDMOND, WA, 98052-6399, US

(73) Assignee: Microsoft Corporation, Redmond, WA (US)

(21) Appl. No.: 11/170,843

(22) Filed: Jun. 30, 2005

(51) Int. Cl. A61K 9/48; A61B 1/00

(52) U.S. Cl. 424/451; 600/102

(57) ABSTRACT

A capsule 1 is adapted to be ingestable by a human or an animal and comprises a location determining device 3 for determining the capsule's 1 location following ingestion.

### TECHNICAL FIELD

This description relates generally to a capsule that is adapted to be ingested by a human or animal for medical treatment and/or diagnosis.

### BACKGROUND

Traditionally, medications and other substances in capsule form have been administered to humans or animals by ingestion of the capsules and the resultant decomposition of the capsules in the body. However, it has proven difficult to control when and where the medication or other substance is released within the body, with any accuracy, which results in less effective treatment than may be desired.

Recently, “intelligent” systems that control the release of a medication or other substance, so that the release takes place at a certain position in the human or animal body, have been developed. These systems include an ingestable capsule and an external positioning and control device that is adapted to communicate with the capsule to determine its location and control release of the medication or other substance from the capsule when it is in a certain predetermined location. An example of such an “intelligent” system is the Enterion® system that is manufactured by Phaeton Research Limited. The Enterion® capsule contains a radioactive tag, a coil and a medication or other substance that is intended to be released in a human or animal body. As the Enterion® capsule moves through the human or animal body, the radioactive tag allows it to be monitored by external equipment, such as a gamma camera. When the capsule has reached a suitable position in the body for releasing the medication or other substance, a magnetic field is applied to the coil by an external source, so that the medication or other substance is released from the capsule to the body. There is also a radio transmitter in the capsule to communicate with external equipment to confirm that the medication or other substance has been transmitted.

One disadvantage of the Enterion® system is that the external equipment that is required for operation of the system is bulky and costly, which means that it is only practical for the system to be used in a clinical environment. Travel to and from a suitable environment, such as a clinic or hospital, can be inconvenient for a patient. In addition, once a patient has traveled to the clinic or hospital, they are required to stay there until the required medication or other substance has been released at a predetermined location in their body. Given that the system is complex, trained specialists are also required to control the system. Furthermore, the inclusion of communication means in the capsule to facilitate contact with the external positioning and control device results in a relatively large capsule which can be difficult to ingest, particularly for children. The communication of the capsule with the external positioning and control device via radio waves is also problematic because the radio signals tend to be attenuated by the bodily fluids through which they pass.

Alternative “intelligent” systems that have been developed include capsules that use different techniques for determining the capsule location within the body, including measurement of the acidity/alkalinity (pH) of an area surrounding the capsule, measurement of the temperature or pressure of an area surrounding the capsule, and imaging. However, these capsules also require external control to determine their location and, if necessary, to release a medication or other substance into the body.

### SUMMARY

The following presents a simplified summary of the disclosure in order to provide a basic understanding to the reader. This summary is not an extensive overview of the disclosure and it does not identify key/critical elements of the invention or delineate the scope of the invention. Its sole purpose is to present some concepts disclosed herein in a simplified form as a prelude to the more detailed description that is presented later.

In accordance with a first aspect of the present invention there is provided a capsule adapted to be ingestable by a human or an animal and comprising a location determining device for determining the capsule's location following ingestion.

The ability of the capsule to determine its own location means that the capsule can be used in the controlled release of substances within a human or animal body so that the release takes place in a desired predetermined location within the body without the need for external control. The predetermined location can be a specific position within the body or a location that exhibits certain characteristics. In addition, the capsule is capable of gathering information using that location data. Such information can be useful in diagnosing medical conditions or analyzing the behavior of a human or animal body in certain circumstances.

In a preferred embodiment of the invention, the location determining device is an accelerometer. Alternatively, the location determining device could be a tilt sensor, an optical device, a temperature measurement device, a pH measurement device, a pressure measurement device, a gyroscope or an audio device.

In a further preferred embodiment, the capsule comprises a data storage device. This allows any data collected by the capsule to be extracted when the capsule is ejected or removed from the area in which it was inserted.

In accordance with a second aspect of the present invention there is provided a method of medical diagnosis, comprising enabling ingestion in a human or animal body of a capsule that has a location determining device for determining the capsule's location within a human or animal body and a data storage device; collecting data from the data storage device of the capsule and analyzing that data to produce a diagnosis.

In accordance with a third aspect of the present invention there is provided a method of treating a medical condition in a human or an animal body, comprising enabling ingestion in a human or an animal body of a capsule that has a location determining device for determining the capsule's location within a human or animal body and a release mechanism for releasing a substance into the human or animal body.

Many of the attendant features will be more readily appreciated as the same becomes better understood by reference to the following detailed description considered in connection with the accompanying drawings.

### DESCRIPTION OF THE DRAWINGS

The present description will be better understood from the following detailed description read in light of the accompanying drawings, wherein:

FIG. 1 is a schematic diagram of a capsule in accordance with a first embodiment of the invention;

FIG. 2 is a circuit diagram of a capsule in accordance with a second embodiment of the invention;

FIG. 3 is a circuit diagram for a system for releasing a substance from the capsule of FIG. 1;

FIG. 4 is a flowchart of the steps that take place when a capsule in accordance with the invention is ingested for the purpose of medical treatment; and

FIG. 5 is a flowchart of the steps that take place when a capsule in accordance with the invention is ingested for the purpose of medical diagnosis.

Like reference numerals are used to designate like parts in the accompanying drawings.

### DETAILED DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

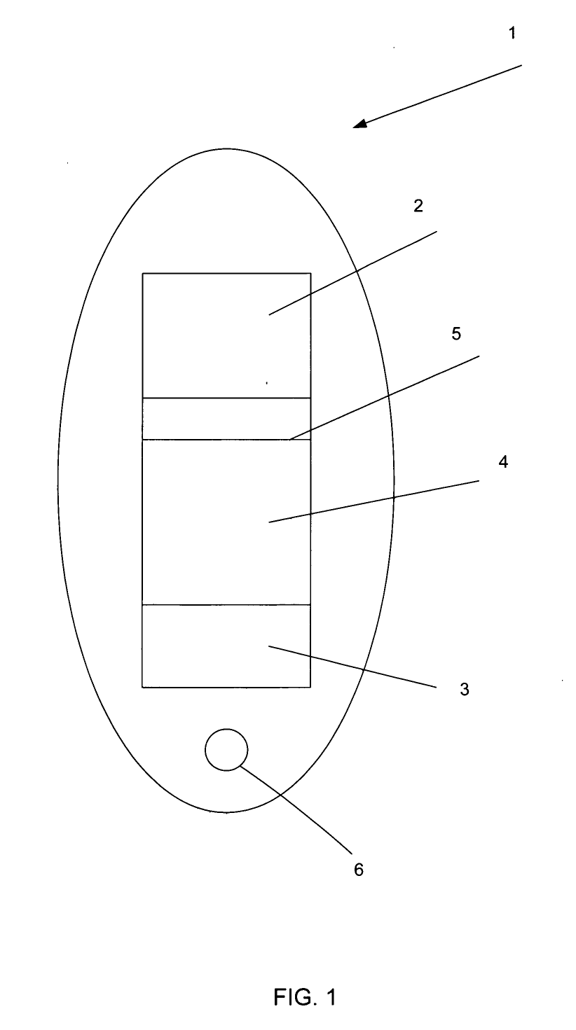

In FIG. 1, a capsule 1 contains a chamber 2 for holding a substance to be released from the capsule 1, a location determining device 3 in the form of an accelerometer, a microprocessor 4 for processing data collected from the location determining device 3 and a release mechanism 5 for releasing the substance from the chamber 2. There is also an energy source in the form of a battery 6 in the capsule 1 to provide power to the accelerometer 3 and microprocessor 4, and if necessary, to the release mechanism 5.

The capsule 1 is made of medical-grade polycarbonate, or any other suitable material, and is between approximately 21 mm and 36 mm long and between 7 mm and 12 mm in diameter. It will be appreciated that the material used to manufacture the capsule 1 will depend largely on the application of the capsule 1. The accelerometer 3 is an Analog Devices chip but any other suitable accelerometer could be used. The microprocessor 4 is a 12F675 processor, although any suitable processor could be used as an alternative. The battery 6 is a medical battery but could be replaced by any other suitable battery, depending on the application of the capsule 1.

When the capsule 1 is inserted in an area in which the substance is to be released, for example, a human body, a timer (not shown) within the capsule starts to record time. In a human body, for example, time would start to be recorded as soon as the capsule was ingested. The timer could be started automatically, for example, by detecting a body temperature of 37° C.; an internal semiconductor temperature sensor would be included to permit temperature measurement. Alternatively, the timer could be activated upon contact with bodily fluids or by a user just prior to ingestion of the capsule 1.

The accelerometer 3 records the acceleration of the capsule in three dimensions at timed intervals as the capsule 1 moves through the body and the timer data and accelerometer data are processed by the microprocessor 4. It would also be possible to record the acceleration in only one or two dimensions.

By using the following equation, the microprocessor 4 can calculate the change in velocity of the capsule 1 corresponding to the recorded acceleration over any given time period:
Change in velocity=acceleration×time.

Once a change in velocity value has been calculated for a specific region by the microprocessor 4, the microprocessor 4 compares that value to a stored standard list of expected values for different locations within the body, so that the position of the capsule 1 can be determined. In the body, velocity varies considerably along the gastrointestinal tract due to varying fluid viscosity at different locations within the tract. Therefore, a certain change in velocity value should indicate that the capsule 1 is at a certain location within the body. The standard list of expected change in velocity values that is stored by the microprocessor 4 is determined empirically and transferred to the microprocessor 4 prior to use of the capsule 1.

When the microprocessor 4 establishes that the capsule 1 is at a certain predetermined location that is desirable for delivering the substance from the capsule 1, it activates the release mechanism 5 so that the substance is released. The predetermined location can be a specific position which is known to be affected by a disease, such as cancer, or a location at which certain characteristics are present, for example, a blockage or excessive bleeding. It will be appreciated that the focussed application of a substance, such as a medication, would be particularly useful in the treatment of certain diseases, which only affect localized areas of the human or animal body. If the drug is released directly at the affected area, it has a higher chance of being effective against the disease. Similarly, if the medication to be administered can harm healthy tissue, it might be desirable to focus the administration to a specific area that requires treatment. Generally, it is also desirable to use as low a dose of medication as possible; a more focussed administration of medication allows does to be kept to a minimum level.

A possible algorithm that could be used by the microprocessor 4 to determine location of the capsule 1 and release a substance and/or process data collected from the accelerometer 3 is:

Determining Location

| Power on; | |
|---|---|
| Time delay for n minutes | ;allow stabilization |
| Test_velocity: | |
| Measure acceleration | |
| Change in velocity = acceleration * time | ;for x & y axis |
| If A< change in velocity <b then test_velocity | ;reading velocity |

to determine viscosity

| Release of a Substance | Diagnosis |
|---|---|
| Heater = on for T secs. | Record data |
| End | End |

Since the velocity at which the capsule 1 moves within certain areas of the body will not always vary to a measurable extent, alternative location determining devices 3 are envisaged. For example, the location determining device 3 could comprise a temperature, pressure or pH recorder to record the temperature, pressure or pH at various locations, respectively. The microprocessor 4 would then compare the recorded values to those in a standard list of expected values of temperature, pressure or pH to determine the location of the capsule 1 in the manner described above.

A further alternative location determining device 3 would be a tilt or angle sensor for measuring the number of times the capsule 1 changes direction as it passes through the body. Given that there are many bends along the gastro-intestinal tract, it should be possible to map data collected from the tilt sensor onto a profile of the tract to determine the capsule's 1 location within that tract. The microprocessor 4 could be pre-programmed to hold the necessary profile data to enable it to carry out this step.

A possible algorithm that could be used by the microprocessor 4 to determine location of the capsule 1 and release a substance and/or process data collected from the tilt sensor 3 is:

Determining Location

| Power on | |
|---|---|
| Time delay for n minutes | ;allow stabilization |
| Count_revolutions: | |
| Read tilt angle = x | |
| Total tilt = total_tilt+ x | ;counting loops of GI tract |

also y axis req

If total_tilt = a then goto heater_on
Goto count_revolutions

| Release of a Substance | Diagnosis |
|---|---|
| Heater = ON for n seconds | Record data |
| End | End |

This embodiment of location determining device 3 would be particularly useful when the specific location of a disease or inflammation was known. For example, if an ulcer was known to be present in the stomach, it would be desirable to refrain from administering a medication until the capsule 1 had reached the stomach.

Similarly, the location device 3 could be a gyroscope and the microprocessor could use data gathered by the gyroscope to determine the location of the capsule 1.

It is possible that the capsule 1 would be shaped so that it only travels in one direction, i.e. it does not turn around as it moves. If so, a tilt sensor would not be a particularly useful location determining device 3.

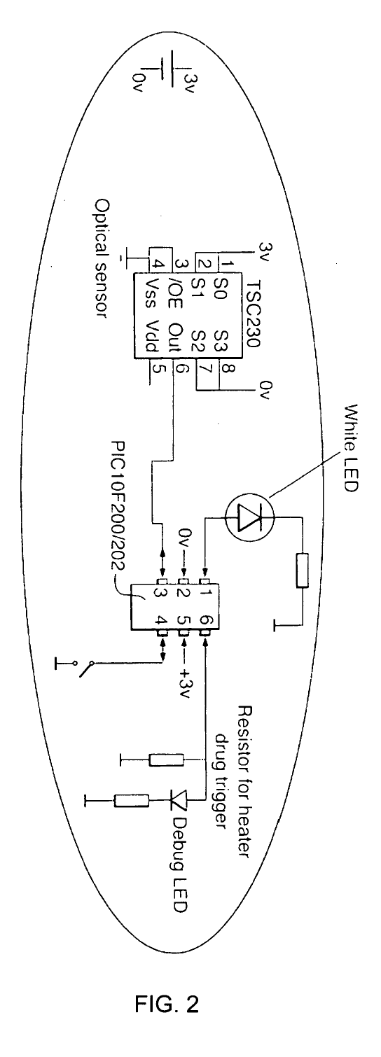

A further suitable location determining device 3 is an optical device that would emit a light signal and record the light reflected back from the area in which the capsule 1 was located, such as a TCS230 chip, marketed by Texas Advanced Optoelectronic Solutions, Inc. The microprocessor 4 would then process the data received from the reflected light and determine whether or not the substance was to be released at a certain point. This embodiment of location determining device 3 would be useful in cases where a medication was to be administered in response to certain characteristics. If the substance was intended to treat a blockage, for example, it would be desirable to release the substance in the vicinity of the blockage. The blockage would be characterized by the amount of light reflected therefrom. An optical device could also be used to indicate an area in which an excessive amount of blood was present, which would have a different light reflection profile than healthy tissue. Preferably, white light would be emitted by the optical device and red, green and blue light would be analyzed following the reflection of the white light. The circuitry for such an optical device is shown in FIG. 2.

Similarly, it would be possible to include a location determining device 3 in the form of a sound-emitting device, the signals of which could be processed by the microprocessor 4 following reflection in order to determine the location of the capsule 1.

Subject to space restrictions and the processing capability of the microprocessor 4, more than one and possibly all of the embodiments of location determining device 3 discussed above could be incorporated in the capsule 1.

The way in which the substance is released from the capsule 1 depends on the area in which the capsule 1 is intended to be used. In some parts of the human body, for example, the release will need to take place in a relatively high pressure environment, which means that the substance will need to be forcibly expelled from the chamber 2 by means of a biasing force or other suitable force. In other circumstances, however, it may be sufficient for the chamber 2 to simply be opened to release the substance.

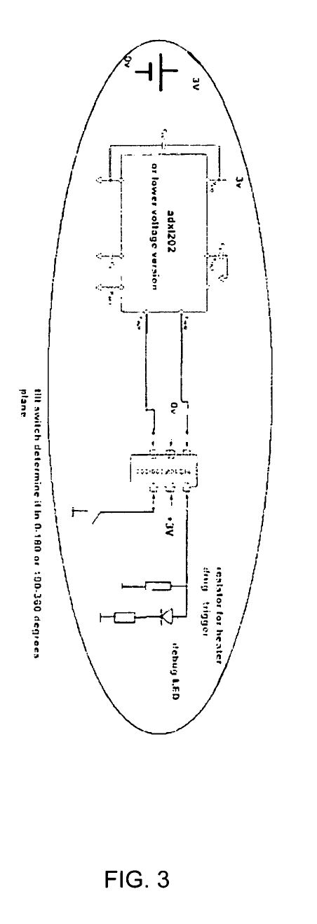

FIG. 3 shows a circuit diagram for one embodiment of release mechanism 5. The release mechanism 5 includes a trigger (not shown) that is activatable to release the substance from the capsule 1 when the trigger is heated to a certain temperature. Heat is applied to the trigger in the form of electricity. Once the microprocessor 4 has determined that the capsule 1 has reached its predetermined location, it supplies the trigger with electricity so that it heats up and releases the substance when it reaches a certain temperature.

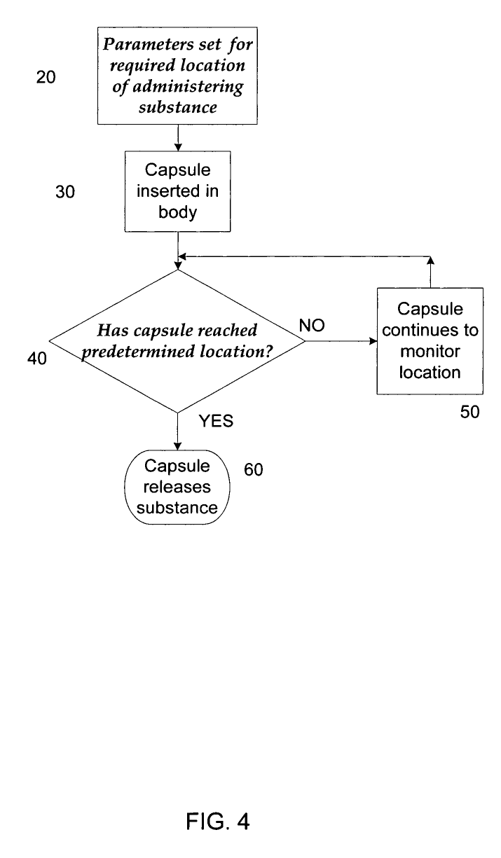

FIG. 4 shows the steps that are followed when the capsule 1 is used to treat a medical condition. At step 20, the capsule 1 is programmed to release a substance at a particular predetermined location within a human or animal body. The programming can take the form of instructions to release a substance at a particular location, for example, in the stomach, or can include additional parameters so that the substance is released where certain characteristics are present, for example, where there is a large amount of blood present. Following ingestion of the capsule 1 by a human or animal at step 30, the capsule 1 travels through the human or animal body and periodically determines its location using the location determining device 3. Each time the capsule 1 determines its location, that location is compared at step 40 with the programmed predetermined location at which the substance is intended to be released. If the comparison does not indicate that the capsule 1 has reached the predetermined location, the capsule 1 continues to determine its location periodically at step 50 until it reaches the predetermined location. Once the capsule 1 has reached the predetermined location, it releases the substance at step 60.

In a further embodiment of the invention, the capsule 1 could also be used as a data collection device, in that it could store data collected on its path for extraction at a later stage. Data collection could be conducted in addition to or as an alternative to releasing a substance. Data extraction could take place following ejection or removal of the capsule 1 from the area in which it was inserted. For example, in the case of using the capsule 1 in the human body, data could be collected from the capsule 1 after it is passed out of the human body. The data collected could be used as a research tool to aid medical diagnosis and also facilitate developing profiles for certain diseases that could later be used in the prevention and treatment of those diseases.

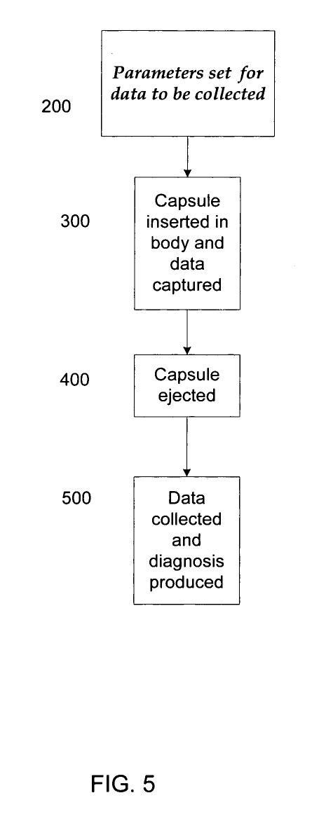

FIG. 5 shows the steps that are followed when the capsule 1 is used to perform a medical diagnosis following data collection. At step 200 the capsule 1 is programmed to collect certain data as it passes through the body and store that data on a data storage device (not shown). The capsule 1 is then inserted in the area in which data is to be collected at step 300. Following collection of the required data at step 400 the capsule 1 is ejected or removed from the body and the data is obtained from the data storage device of the capsule 1 and analyzed at step 500.

Where the capsule 1 is inserted in an area which is itself moving, a further fixed data collection device, such as a further accelerometer, could be used in combination with the data collected from the capsule 1 in order to calculate the relative movement of the area and the capsule 1 itself. In the case of the human body, it might be useful to collect and analyze data from the capsule 1 when the body surrounding the capsule is moving at a considerable speed or is subjected to a considerable force. If this is the case, the data collected from the fixed data collection device needs to be combined with the data extracted from the capsule 1. Such analysis could be useful in preventing disease or other conditions, such as stress. It might also be useful to monitor how certain diseases are affected by external factors, such as driving a car or flying, so that the effects of those diseases can be mitigated.

It will be appreciated that the applications of this invention are far-reaching. As discussed above, the capsule 1 could be used in medical and veterinary applications to administer medications and other therapeutic substances to specific locations within the human or animal body, respectively. Administering medications and other therapeutic substances in this way would achieve more efficient and more effective treatment of diseases and isolated injuries and inflammations, such as ulcers. To this extent, the invention would be equally applicable on a nano-level.

From the above description of the invention, those skilled in the art to which the present invention relates will perceive improvements, changes and modifications. Numerous substitutions and modifications can be undertaken without departing from the true spirit and scope of the invention. For example, the release mechanism 5 could comprise a device for releasing a solvent so that an outer shell of at least part of the capsule 1 would dissolve to release the substance. Alternatively, the release mechanism 5 could be adapted to disintegrate to release the substance.

It will also be appreciated that the invention has numerous non-medical applications, including clearing blockages in sewage and other plumbing systems by means of chemicals, administering horticultural products and releasing product ingredients at certain stages in a production process. Any industrial application that requires release of a substance at a specific, relatively inaccessible location could avail of the invention.

1. A capsule adapted to be ingestable by a human or an animal and comprising a location determining device for determining the capsule's location following ingestion.

2. A capsule as claimed in claim 1, wherein the location device comprises an accelerometer.

3. A capsule as claimed in claim 2, wherein the accelerometer is capable of measuring acceleration in three directions.

4. A capsule as claimed in claim 1, wherein the location device comprises an optical device.

5. A capsule as claimed in claim 4, wherein the optical device is adapted to emit white light.

6. A capsule as claimed in claim 1, wherein the capsule is capable of containing a substance.

7. A capsule as claimed in claim 6, further comprising a release mechanism for releasing the substance.

8. A capsule as claimed in claim 6, wherein the substance is a medication.

9. A capsule as claimed in claim 1, wherein the capsule additionally comprises a processor.

10. A capsule as claimed in claim 1, wherein the capsule is programmable prior to use.

11. A capsule as claimed in claim 1, wherein the capsule additionally comprises a data collection device.

12. A capsule as claimed in claim 1, wherein the capsule additionally comprises a data storage device.

13. A capsule as claimed in claim 1, wherein the capsule further comprises a timer.

14. A method of medical diagnosis, comprising enabling ingestion in a human or animal body of a capsule that has a location determining device for determining the capsule's location within a human or animal body and a data storage device; collecting data from the data storage device of the capsule and analyzing that data to produce a diagnosis.

15. A method as claimed in claim 14, wherein the data collected from the capsule is combined with data collected from a further source to produce the diagnosis.

16. A method as claimed in claim 14, further comprising programming the capsule prior to enabling its ingestion.

17. A method of treating a medical condition in a human or an animal body, comprising enabling ingestion in a human or an animal body of a capsule that has a location determining device for determining the capsule's location within a human or animal body and a release mechanism for releasing a substance into the human or animal body.

18. A method as claimed in claim 1 7, further comprising programming the capsule prior to enabling its ingestion.
